(19) United States
(12) Patent Application Publication
(10) Pub. No.: US 2025/0169945 A1
(43) Pub. Date: May 29, 2025

(54) PROSTHETIC HEART VALVE

(71) Applicant: Medtronic, Inc., Minneapolis, MN (US)

(72) Inventors: Nathan B. Wiemeyer, Windsor, CA (US); Elliot C. Schmidt, Minneapolis, MN (US); Justin Goshgarian, Santa Rosa, CA (US)

(21) Appl. No.: 18/841,368

(22) PCT Filed: Feb. 16, 2023
(86) PCT No.: PCT/IB2023/051383

§ 371 Date: Aug. 24, 2024

Related U.S. Application Data
(60) Provisional application No. 63/268,575, filed on Feb. 25, 2022.

(51) Int. Cl. A61F 2/24

(52) U.S. Cl. CPC A61F 2/2418; A61F 2/2436; A61F 2220/0008

(57) ABSTRACT

In some examples, a prosthetic device is configured to expand radially outward to position a valve assembly to control blood flow through an annulus of a heart valve. The prosthetic device supports a plurality of imaging markers around a perimeter defined by the prosthetic device. In examples, the prosthetic device includes an anchoring member configured to expand to engage the annulus of the heart valve. In examples, the anchoring member defines the perimeter. The prosthetic device may support the imaging markers to radially extend inwards and/or laterally extend in an upstream and/or downstream direction of the prosthetic device to displace the imaging markers from tissues within the heart when the prosthetic device engages the annulus of the heart valve.

### TECHNICAL FIELD

The present disclosure relates generally to prosthetic heart valve devices and associated systems and techniques.

### BACKGROUND

Human hearts can suffer from various diseases or conditions related to heart valves. One method of treatment includes replacement of the heart valve by implanting a prosthetic heart valve device into the heart in place of a native heart valve (e.g., a mitral valve or tricuspid valve). Another method of treatment includes repair, bypassing, or replacement of a previously implanted prosthetic heart valve device. In some cases, one or more prosthetic heart valve devices may be implanted percutaneously using valve delivery devices. In some cases, a prosthetic heart valve device may be sheathed within a capsule to allow for the percutaneous delivery via a catheter, with the prosthetic heart valve device assuming a relatively small cross-sectional dimension in the fully sheathed configuration. Once delivered and placed in the target site, the prosthetic heart valve device may be unsheathed to expand to assume a larger cross-sectional dimension. During and/or following implantation, alignment of the prosthetic heart valve device within the patient may be evaluated using imaging techniques.

### SUMMARY

In some examples, the disclosure is directed to a prosthetic device such as a prosthetic heart valve device configured to operate as a heart valve within a heart of a patient. The prosthetic device is configured to expand radially outward to position a valve assembly that is configured to control blood flow through an annulus of the heart valve. The prosthetic device supports a plurality of imaging markers around a perimeter defined by the prosthetic device to aid in imaging the prosthetic device within the patient, such that a clinician may assess a position and/or orientation of the prosthetic device. The prosthetic device is configured to support the imaging markers in a manner enhancing the visual clarity of a resulting image. For example, the prosthetic device may support the imaging markers to radially extend inwards and/or laterally extend in an upstream and/or downstream direction of the prosthetic device to, for example, provide beneficial imaging angles to the imaging system and/or assist is displacing the imaging markers from tissues within the heart.

In examples, a prosthetic device comprises: an anchoring member configured to engage an annulus of a heart valve of a heart, wherein the anchoring member is configured to cause a valve axis defined by the prosthetic device to pass through the annulus when the anchoring member engages the annulus; a valve support mechanically supported by the anchoring member and surrounding the valve axis, wherein the valve support is configured to define a flow path from an inflow region of the valve support to an outflow region of the valve support; a valve assembly mechanically supported by the valve support within the flow path, wherein the valve assembly is configured to allow a blood flow through the flow path; and a plurality of imaging markers mechanically supported by the prosthetic device around a perimeter defined by the prosthetic device, wherein the perimeter surrounds the valve axis.

In examples, a method comprises: expanding, by a prosthetic device, at least a portion of the prosthetic device in proximity to an annulus of a heart valve of a heart, wherein the prosthetic device includes an anchoring member configured to engage the annulus, and wherein the anchoring member is configured to cause a valve axis defined by the prosthetic device to pass through the annulus when the anchoring member engages the annulus; supporting, by the anchoring member, a valve support surrounding the valve axis, wherein the valve support is configured to define a flow path from an inflow region of the valve support to an outflow region of the valve support; supporting, by the valve support, a valve assembly within the flow path, wherein the valve assembly is configured to allow a blood flow through the flow path; and supporting, by the prosthetic device, a plurality of imaging markers around a perimeter defined by the prosthetic device, wherein the perimeter surrounds the valve axis.

The details of one or more examples are set forth in the accompanying drawings and the description below. Other features, objects, and advantages will be apparent from the description and drawings, and from the claims.

### BRIEF DESCRIPTION OF DRAWINGS

FIG. 1A is a conceptual diagram illustrating an example delivery system including a prosthetic device in a delivery configuration.

FIG. 1B is a conceptual diagram of the delivery system of FIG. 1A with the prosthetic device in an expanded configuration.

FIG. 2 is a conceptual diagram of an example prosthetic device supporting a plurality of echogenic markers.

FIG. 3A is a conceptual diagram illustrating a cross-section of an example prosthetic device supporting a plurality of echogenic markers, the cross-section being taken through an axis L of the prosthetic device.

FIG. 3B is a conceptual diagram illustrating a top plan view of the example prosthetic device of FIG. 3A.

FIG. 4 is a conceptual diagram illustrating a cross-section of an example prosthetic device supporting a plurality of echogenic markers defining a curvature, the cross-section being taken through an axis L of the prosthetic device.

FIG. 5 is a conceptual diagram of an example prosthetic device supporting a plurality of echogenic markers around a defined perimeter.

FIG. 6 is a conceptual diagram of an example imaging marker in a compressed state.

FIG. 7 is a conceptual diagram of the imaging marker of FIG. 6 in an extended state.

FIG. 8 is a conceptual diagram illustrating a perspective view of the imaging marker of FIG. 6 and FIG. 7 in an extended state.

FIG. 9 is a flowchart illustrating an example technique of using a prosthetic device.

### DETAILED DESCRIPTION

This disclosure describes a prosthetic device such as a prosthetic heart valve device configured to operate as a heart valve within a heart. The prosthetic device is configured to establish a relatively compact delivery configuration for delivery to a heart of a patient. The prosthetic device is configured such that a clinician may cause the prosthetic device to expand from the delivery configuration to an expanded configuration when the prosthetic device is positioned in proximity to a native heart valve, such that the prosthetic device may engage tissue of the heart to position a valve assembly substantially within an annulus of the native heart valve. The prosthetic device supports a plurality of imaging markers (e.g., echogenic markers and/or radiopaque markers) configured to aid in imaging the prosthetic device within the patient using an imaging system, such that a clinician may assess a position and/or orientation of the prosthetic device during, for example, an implantation procedure, a post-operative check, a check-up of the patient, or at other times.

The prosthetic device is configured to support the imaging markers in a manner that enhances the visual clarity of a resulting image. In examples, the prosthetic device supports the imaging markers such that the imaging markers extend radially extend inwards to, for example, provide beneficial imaging angles to the imaging system. The prosthetic device may support the imaging markers such that the imaging markers laterally extend in an upstream and/or downstream direction of the prosthetic device. The radial and/or lateral extension may assist is displacing the imaging marker from tissues within the heart, enhancing the visual clarity of the image markers relative to tissue interfaces. In examples, the prosthetic device may support the imaging markers in a manner allowing for a reduction in the size of a delivery system (e.g., a delivery capsule) configured to deliver the prosthetic device to the heart. The size reduction may broaden the population of patients eligible to receive the prosthetic device, since the anatomical dimensions and/or shape of the some heart structures (e.g., the right ventricle) in some patients may be too short or shaped in a way that might limit navigation of the capsule to the annulus of the heart valve the prosthetic device is configured to replace.

During the implantation of the prosthetic device within a heart, the clinician may position the delivery capsule (using a delivery system) in the vicinity of an annulus of the native heart valve in preparation for unsheathing the prosthetic device. The prosthetic device may be configured such that, when the capsule unsheathes the prosthetic device to allow for expansion, the prosthetic device grips the annular wall of the native heart valve to substantially secure the prosthetic device within the heart. For example, the prosthetic device may include a fixation structure including one or more fixation elements (e.g., barbs, hooks, cleats, tines, and the like) configured to engage with the annular wall when the prosthetic device expands. Consequently, during and following the transition of the prosthetic device to the expanded configuration, alignment of the prosthetic device with the annular wall is typically evaluated using an imaging system, in order to allow a clinician to evaluate the relative positions of the prosthetic device and the native heart valve of the patient.

The prosthetic device includes a plurality of imaging markers configured to aid in identifying the prosthetic device within the patient using an imaging system, such as an ultrasound system. The imaging markers may be configured to enhance the image of the prosthetic device captured by the imaging system, such that an approximate location of the prosthetic device within the heart of the patient may be assessed by the clinician. In examples, the imaging markers may be located at one or more fixed locations on the prosthetic device, such that an approximate orientation of the prosthetic device within the heart may be assessed using the imaging system. Hence, the imaging markers may enhance the ability of a clinician to assess the position and/or orientation of the prosthetic device within the heart of the patient during and/or following the transition of the prosthetic device to the expanded configuration.

In some examples, the imaging markers are configured to reflect and or otherwise interact with energy transmitted by the imaging system. The imaging system may be configured to transmit energy (e.g., sound, x-rays, and the like) and produce an image based on the return of at least some portion of the transmitted energy to the image device. In examples, the imaging markers are configured to interact with the transmitted energy to enhance a resulting image of the image markers produced by the imaging system. For example, the image markers may be configured to interact with the transmitted energy such that the imaging system provides a contrast and/or other distinction (e.g., visual distinction) between the imaged image markers and one or more structures of the heart. The contrast and/or other distinction may assist the clinician in assessing a position and/or orientation of the prosthetic device.

In some examples, the imaging system may be an ultrasound device configured to utilize an ultrasound technique to produce an image of the prosthetic device within the heart of the patient. In an ultrasound technique, acoustic wave energy generated by an ultrasound probe (which is sometimes located in the esophagus of a patient) reflect off of the prosthetic device back to the ultrasound probe. Reflected wave energy captured by the probe may be sensed and used to generate an image displaying the position of the prosthetic device within the patient. In examples, the prosthetic device includes imaging markers including echogenic surfaces configured to reflect acoustic waves back to an ultrasound probe, such that echogenic visibility of the prosthetic device is improved.

The prosthetic device may be configured to spatially position the imaging markers such that the spatial positioning indicates an orientation of the prosthetic device within the heart. For example, the prosthetic device may be configured to allow blood flow generally along a valve axis from an inflow region of the prosthetic device to an outflow region of the prosthetic device when the prosthetic device is in the expanded configuration. The prosthetic device may be configured such that the relative position of one or more imaging markers is defined relative to the valve axis (e.g., in a perimeter around the valve axis) and/or a portion of the prosthetic device when the prosthetic device is in the expanded configuration. Hence, the spatial positioning of the imaging markers may assist a clinician in assessing the orientation the valve axis relative to other structures of the heart when the clinician views an image of the prosthetic device within the heart. In like manner, the relative position of one or more imaging markers may be defined relative to other portions of the prosthetic device (e.g., a fixation structure, a valve assembly, and/or other portions), such that the spatial positioning of the imaging markers may assist a clinician in assessing the orientation of other portions of the prosthetic device relative to other structures of the heart.

For example, in some examples, the prosthetic device includes a plurality of imaging markers spatially positioned around a perimeter defined by the prosthetic device in the expanded configuration. The prosthetic device may, for example, define the perimeter around the valve axis. In examples, the prosthetic device defines the perimeter around a flow path from the inflow region of the prosthetic device (e.g., a region fluidically coupled to an atrium of a heart) to an outflow region of the prosthetic device (e.g., a region fluidically coupled to a ventricle of the heart). In some examples, the perimeter defined by the prosthetic device is substantially perpendicular to the valve axis defined by the prosthetic device when the prosthetic device is in the expanded state. The spatial positioning of the imaging markers around the defined perimeter may assist a clinician in assessing the orientation of the prosthetic device relative to other structures of the heart when the clinician views an image of the prosthetic device within the heart.

In some examples, the prosthetic device includes a fixation structure configured to grip an annular wall of a native heart valve when the prosthetic device is in an expanded configuration to help secure the prosthetic device in an annulus of the native heart valve. The prosthetic device may be configured to cause a valve assembly to position within or in the vicinity of the annulus when the fixation structure grips the annular wall. The valve assembly may be configured to allow blood to flow along a flow path from the inflow region of the prosthetic device to the outflow region of the prosthetic device. In some examples, the valve axis passes through the valve assembly. The prosthetic device (e.g., an anchoring member, a valve support, or another portion of the prosthetic device) may define the perimeter substantially surrounding the flow path, such that the spatial positioning of the imaging markers around the defined perimeter may assist a clinician in assessing the orientation of the flow path when the clinician views an image of the prosthetic device within the heart.

In examples, the plurality of imaging markers is a series of individual imaging markers arranged around the defined perimeter. An individual imaging marker may be a discrete image marker configured to interact with the transmitted energy of an imaging system. The individual imaging markers can be separated from an adjacent imaging marker. For example, the individual image marker may substantially reside on the defined perimeter and be separated from a nearest neighboring image marker by an arc-length of the defined perimeter. As an example, an individual image marker may be separated by an arc-length defined by a subtending angle of about 30 degrees, 45 degrees, 90 degrees, or some other subtending angle. Stated similarly, each individual imaging marker in the plurality of image markers may define a closed boundary defining one or more spatial parameters of the individual image marker, such as a length, a surface area, a volume, and/or another spatial parameter. Individual imaging markers may be arranged around the perimeter defined by the prosthetic device such that each closed boundary of an imaging marker is displaced from every other closed boundary of the other imaging markers.

The plurality of imaging markers (e.g., echogenic markers) may be configured to enhance the imaged visibility of the prosthetic device while accommodating the space considerations present when operating in a human heart. For example, in some examples, the prosthetic device may be configured such that the defined perimeter is substantially surrounded by the annular wall of a native heart valve when the fixation structure grips the annular wall. The prosthetic device may mechanically support one or more imaging markers such that at least some or all of the imaging markers extend substantially from the defined perimeter in an upstream direction of the prosthetic device toward an upstream portion of the prosthetic device (e.g., extend in a direction opposite the blood flow through the valve assembly). In examples, an imaging marker may include a fixed end mechanically coupled to a portion of the prosthetic device (e.g., an anchoring member, a valve support, or another portion) and a free end substantially opposite the fixed end. The prosthetic device may mechanically support the plurality of imaging markers such that a plurality of free ends extends in the upstream direction of the prosthetic device, such that a radial extension of the imaging markers beyond the defined perimeter is limited. Further, this may allow the plurality of imaging markers to position and/or extend in a direction away from tissue interfaces (e.g., the annular wall), enhancing the visual clarity of the image markers relative to the tissue interfaces and providing for clarity and/or enhanced assessment of a prosthetic device deployment.

In some examples, one or more of the imaging markers are configured to radially extend inwards toward the valve axis when supported by the prosthetic device. For example, an imaging marker may have an angled, curved, or curvilinear portion configured to extend inward toward the valve axis from the defined perimeter. This may provide beneficial imaging angles to an imaging system when the prosthetic device is positioned within and/or in the vicinity of an annulus of a native heart valve. This may also assist is displacing at least some portion of the imaging marker from tissues within the heart, enhancing the visual clarity of the image markers relative to the tissue interfaces and providing for clarity and/or enhanced assessment of a prosthetic device deployment.

Any of the imaging marker configurations described herein may be used in combination with each other. That is, the plurality of imaging markers of the prosthetic device can include imaging markers extending in similar directions and/or in different directions.

The reduction in the extension of the imaging markers may enable a reduction in a length of the prosthetic device in the delivery configuration. The reduction in the length of the prosthetic device in the delivery configuration may enable a reduction in the required length of a capsule configured to carry the prosthetic device to a treatment site. A reduced length of the capsule may ease navigation and/or maneuvering of the capsule through a patient's vasculature. For example, when the prosthetic device is configured to replace a native tricuspid valve, a shorter capsule length may ease the navigation and/or maneuvering of the capsule through shorter and more difficult anatomical structures such as chordae, papillary muscles, and structures of the right ventricle. The shorter capsule length may broaden the population of patients eligible to receive transcatheter tricuspid valve replacement, since the anatomical dimensions and/or shape of the right ventricle in some patients may be too short or shaped in a way that might limit navigation of the capsule to the annulus of the native heart valve the prosthetic device is configured to replace.

In examples, the disclosure provides a prosthetic device configured to enhance the visibility (e.g., echogenic visibility) of the prosthetic device to an imaging system when the prosthetic device is within the heart of a patient. In some example, the imaging system is an ultrasound probe configured to produce an image using acoustic energy. The prosthetic device may include a plurality of imaging markers (e.g., echogenic markers) shaped to catch and reflect acoustic waves at an angle substantially determined by the location of the ultrasound probe during a valve replacement procedure (e.g., when the ultrasound probe is located within or in the vicinity of the esophagus of a patient). The configuration of the imaging markers may enable a reduction in the required length of a capsule configured to carry the prosthetic device to a treatment site. Additionally, the plurality of imaging markers may be configured to position away from away from tissue interfaces, providing for clarity and/or enhanced assessment of prosthetic device deployment.

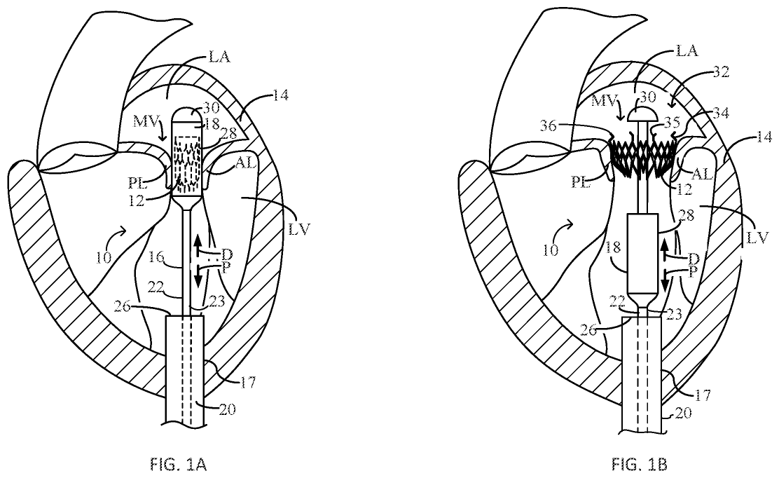

FIG. 1A illustrates a portion of an example delivery system 10 for delivering an example prosthetic device 12 (“device 12”) to a target site within a heart 14. Delivery system 10 includes a delivery catheter 16 supporting a capsule 18, with delivery catheter 16 extending from a lumen defined by a guide catheter 20. FIG. 1A illustrates device 12 in a delivery configuration within capsule 18, where capsule 18 constrains device 12 against radial expansion. FIG. 1B illustrates delivery system 10 with device 12 an expanded configuration, where capsule 18 has been withdrawn such that capsule 18 provides substantially no constraint on the radial expansion of device 12.

For the purpose of illustration, FIG. 1A and FIG. 1B illustrate delivery system 10 positioning the device 12 in a native mitral valve MV of heart 14 using a trans-apical delivery approach. In some examples, device 12 may be configured to be placed in a native tricuspid valve using a transcatheter percutaneous delivery system. Other approaches may be utilized, such as a trans-septal delivery approach.

Referring to FIG. 1A, guide catheter 20 is positioned in a trans-apical opening 17 to provide access to the left ventricle LV. Delivery catheter 16 extends through guide catheter 20 such that a distal portion 22 of a catheter body 23 projects beyond a distal end 26 of guide catheter 20. Capsule 18 may then be positioned between a posterior leaflet PL and an anterior leaflet AL of mitral valve MV. Using a control unit (not pictured), catheter body 23 may be moved in the distal direction (as indicated by arrow D), the proximal direction (as indicated by arrow P), and/or rotated along a longitudinal axis of catheter body 23 to position capsule 18 at a desired location and orientation within the opening of mitral valve MV (or another native heart valve within heart 14).

At the target location, capsule 18 (e.g., capsule housing 28) may be at least partially retracted (e.g., in the proximal direction P) to deploy device 12 from capsule 18. Delivery system 10 may be configured such that withdrawal of capsule housing 28 displaces at least some portion of capsule housing 28 from device 12, such that device 12 may is free to expand into the expanded configuration. In some examples, capsule 18 may include an end cap 30 at the distal end of a capsule housing 28 of capsule 18. In some examples, capsule 18 may be open-ended at the distal end of capsule housing 28. In some examples, capsule 18 may be configured to circumferentially surround at least part of device 12. In the illustrated example of FIG. 1B, device 12 may be deployed from capsule 18 by drawing capsule housing 28 proximally (e.g., further into the left ventricle LV) and, optionally, moving end cap 30 distally (e.g., further into the left atrium LA). As device 12 exits capsule housing 28, device 12 may expand radially to secure the device 12 in an annulus of a native heart valve (e.g., the annulus of mitral valve MV).

As will be discussed further, device 12 includes plurality of imaging markers 32, such as imaging marker 34, imaging marker 35, and imaging marker 36 (“imaging markers 34, 35, 36”). Imaging markers 34, 35, 36 may be configured to provide imaging feedback (e.g., echogenic feedback) to assist in positioning device 12 at a target location within heart 14 before or during deployment of device 12. Although discussed below mainly with reference to imaging marker 34, descriptions below may additionally describe imaging marker 35, imaging marker 36, of any other imaging markers in the plurality of imaging markers 32.

Imaging marker 34 is configured to reflect and or otherwise interact with energy transmitted by an imaging system (not shown) configured to transmit energy (e.g., sound, x-rays, and the like) to produce an image. In examples, imaging marker 34 is configured to interact with the transmitted energy to enhance a visual distinction from one or more structures of the heart 14 in the resulting image. The visual distinction enabled by imaging marker 34 may assist a clinician in assessing a position and/or orientation of device 12 during and/or following a deployment of device 12 within heart 14. In the illustrated example of FIG. 1B, the plurality of imaging markers 32 and/or imaging markers 34, 35, 36 may not be drawn to scale.

In examples, device 12 is configured to mechanically support imaging marker 34 in a manner that helps to limit or even prevent the extension of imaging marker 34 into a chamber of heart 14 (e.g., the left atrium LA) when device 12 is in the expanded configuration and implanted to extend through an annulus of a native heart valve. Limiting the extension of imaging marker 34 reduce a length of device 12 when in the delivery configuration, and may enable a reduction in the length of capsule 18. A reduced length of capsule 18 may ease navigation and/or maneuvering of the capsule. In some examples, where capsule 18 and/or distal portion 22 of catheter body 23 define a relatively stiff section of delivery catheter 16, the reduced length of capsule 18 may allow a length of the relatively stiff section to be reduced by up to 5 millimeters (mm), 8 mm, 10 mm, or more. This may increase the maneuverability of delivery catheter 16 within heart 14, opening certain medical procedures to a wider patient population. For example, percutaneous transcatheter replacement of a tricuspid valve may involve turning capsule 18 over an angle of about ninety degrees for placement. A reduced length of capsule 18 and the relatively stiff section of delivery catheter 16 may be of benefit to patients who might otherwise be candidates for a medical procedure (e.g., transcatheter tricuspid valve replacement), but have an anatomy that will not accommodate a catheter system with a relatively long stiff section.

The examples provided are described herein with reference to devices, systems, and methods for positioning and deploying a prosthetic heart valve to a native mitral valve or tricuspid valve. However, other applications and other embodiments in addition to those described herein are within the scope of the present technology. For example, at least some embodiments of the present technology may be useful for delivering and deploying prosthetics to other native valves such as the aortic valve or pulmonary valve.

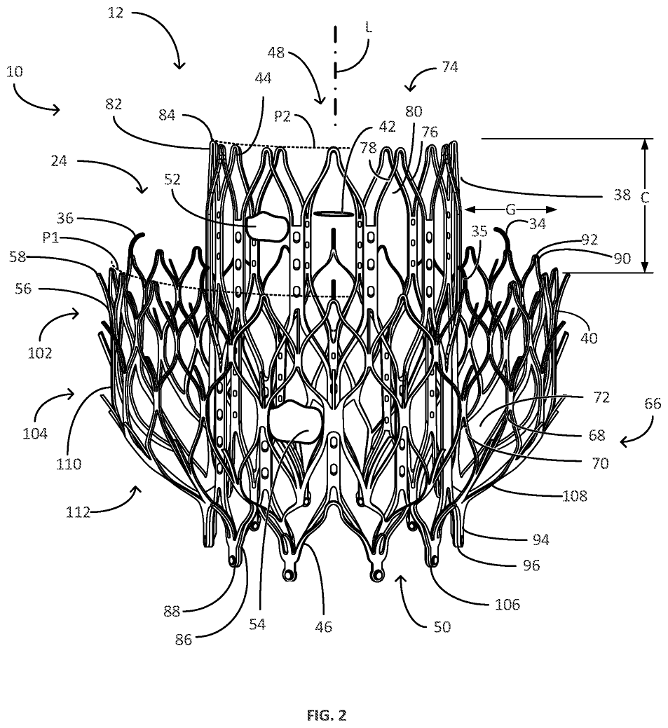

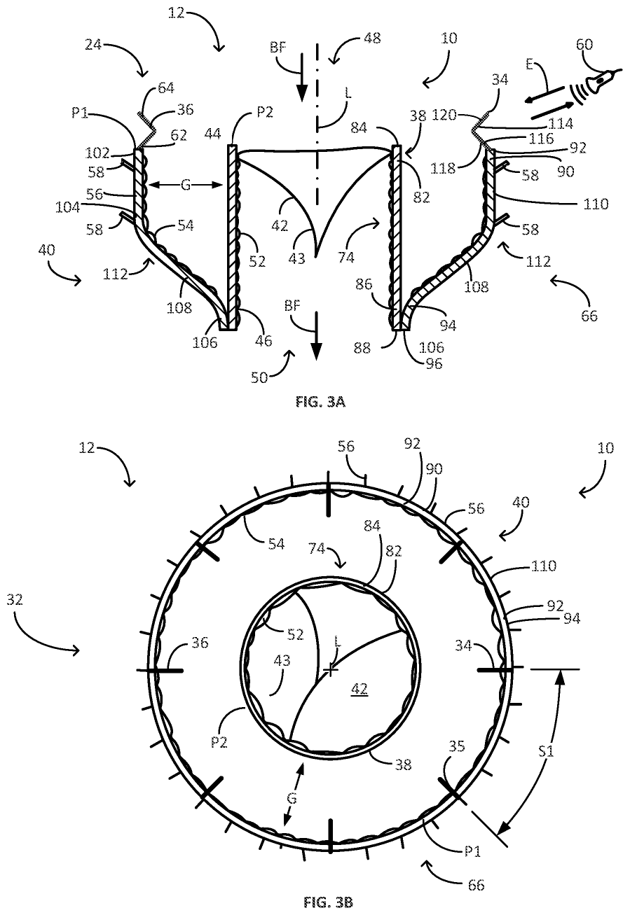

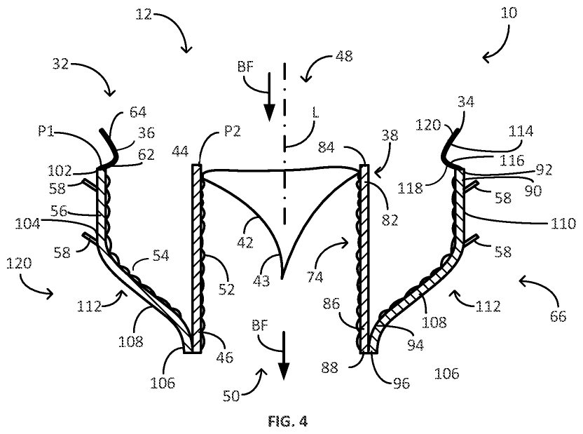

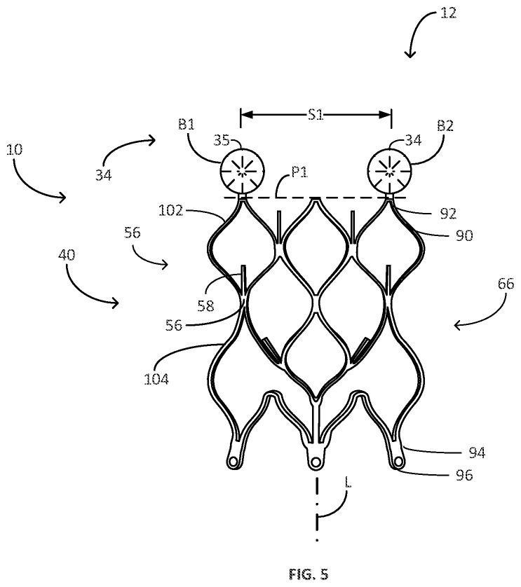

FIG. 2 illustrates a perspective view of portions of device 12 illustrated in an expanded configuration. FIG. 3A is a cross-sectional view of device 12 in an expanded configuration, with the cross-section taken through valve axis L. FIG. 3B is a top plan view of device 12 in an expanded configuration supporting a plurality of imaging markers 24. FIG. 4 is a cross-sectional view of device 12 in an expanded configuration supporting a plurality of imaging markers 24 having a curved shape, with the cross-section taken through valve axis L. FIG. 5 illustrates a portion of an anchoring member 40 supporting a portion of the plurality of imaging markers 24. Device 12 includes a valve support 38, anchoring member 40 attached to the valve support 38, and a prosthetic valve assembly 42 (“valve assembly 42”) within the valve support 38. Anchoring member 40 may be coupled to (e.g., attached) to valve support 38. Valve assembly 42 may be coupled to valve support 38 (as illustrated, for example, in FIG. 3A and/or FIG. 3B). In examples, valve support 38 mechanically supports valve assembly 42. In some examples, device 12 defines a valve axis L intersecting valve assembly 42. In some examples, valve support 38 and/or anchoring member 40 surround some portion of valve axis L.

In examples, device 12 (e.g., anchoring member 40) is configured to expand radially outward from a delivery configuration (e.g., when housed within capsule 18 (FIG. 1A)) to the expanded configuration depicted in FIG. 1B, FIG. 2, FIG. 3A-3B, and FIG. 4. Device 12 may be configured to expand radially outward to substantially grip an annulus of a native heart valve when, for example, device 12 transitions from the delivery configuration to the expanded configuration. Device 12 (e.g., anchoring member 40) may be configured to expand radially to accommodate (e.g., substantially conform to) a perimeter of the annulus of the native heart valve when device 12 is in the expanded configuration. In examples, anchoring member 40 is configured to expand radially outward from valve support 38 to establish a gap G between anchoring member 40 and valve support 38. Gap G may define a radial displacement relative to valve axis L. In some examples, the radial displacement is substantially perpendicular to valve axis L. In some examples, anchoring member 40 is configured to define an anchoring delivery radius relative to valve axis L when device 12 is in the delivery configuration and an anchoring expanded radius relative to valve axis L when device 12 is in the expanded configuration, and the anchoring delivery radius is less than the anchoring expanded radius.

In examples, valve support 38 is a substantially rigid member configured to retain its shape (e.g., define a substantially constant radius around valve axis L) when anchoring member 40 expands radially outward. In some examples, valve support 38 may be configured to expand radially outward when anchoring member 40 expands radially outward. In some examples, valve assembly 42 may be configured to expand radially when valve support 38 and/or anchoring member 40 expands radially.

Referring to FIG. 3A, valve support 38 includes an inflow region 44 and an outflow region 46. In examples, device 12 defines a downstream direction (e.g., the direction of the arrows BF) extending from the inflow region 44 toward the outflow region 46 and an upstream direction (e.g., substantially opposite the direction of arrows BF) extending from outflow region 46 toward inflow region 44. The downstream direction and/or upstream direction may be substantially parallel to valve axis L. In examples, inflow region 44 defines a inlet 48 of valve support 38 (“valve support inlet 48”). Outflow region 46 may define an outlet 50 of valve support 38 (“valve support outlet 50”). In examples, device 12 is configured to define a flow path for blood flow (BF) from valve support inlet 48 to valve support outlet 50.

Valve assembly 42 is configured to be supported within the valve support 38 to allow blood to flow through device 12 in the downstream direction (e.g., from valve support inlet 48 to valve support outlet 50). In examples, valve assembly 42 is configured to allow blood flow through device 12 in the downstream direction, but limit and/or substantially prevent blood flow through device 12 in the upstream direction. In examples, device 12 is configured such that valve assembly 42 controls substantially all blood flow through device 12 when device 12 is in the expanded configuration and positioned within an annulus of a native heart valve.

For example, device 12 may include a first sealing member 52 configured define the flow path through valve support 38. First sealing member 52 may be configured to substantially direct blood flow from valve support inlet 48 to valve support outlet 50. In examples, first sealing member 52 is configured to limit and/or substantially prevent a flow of blood through valve support 38 in a radial direction away from valve axis L. Device 12 may include a second sealing member 54 configured to limit and/or substantially prevent a flow of blood through anchoring member 40. Device 12 may be configured such that, when device 12 is in the expanded configuration and positioned within an annulus of a native heart valve, first sealing member 52 and second sealing member 54 cause substantially all blood flow (e.g., all or nearly all to the extent permitted by the material from which sealing members 52, 54 are formed) passing through device 12 to flow through valve assembly 42. In examples, when device 12 is in the expanded configuration and positioned within an annulus of a native heart valve, first sealing member 52, second sealing member 54, and valve assembly 42 allow blood flow through device 12 in the downstream direction (e.g., from inflow region 44 to outflow region 46), while limiting and/or substantially preventing blood flow through device 12 in the upstream direction e.g., from outflow region 46 to inflow region 44). Device 12 may be configured such that, when device 12 is in the expanded configuration and positioned within an annulus of a native heart valve, inflow region 44 is fluidically coupled to a first chamber (e.g., an atrium) of heart 14 (FIG. 1), and outflow region 46 is fluidically coupled to a second chamber (e.g., a ventricle) of heart 14.

Device 12 includes a fixation structure 56 configured to engage with tissue to help secure device 12 in a heart of a patient. For example, fixation structure 56 can be configured to grip an annular wall of a native heart valve when device 12 is positioned within an annulus of the native heart valve and device 12 is in the expanded configuration. Fixation structure 56 may be configured to secure device 12 within the annulus of the native heart valve, such that valve assembly 42 may allow blood to flow from inflow region 44 through outflow region 46. Device 12 may be configured to cause valve assembly 42 to substantially position within or in the vicinity of the annulus when fixation structure 56 grips the annular wall, such that valve assembly 42 allows blood to flow along a flow path substantially parallel to valve axis L (e.g., from inflow region 44 to outflow region 46). In examples, fixation structure 56 includes a plurality of fixation elements 58 (e.g., barbs, hooks, cleats, tines, or other elements) configured to engage tissue of heart 14. In examples, the plurality of fixation elements 58 are configured to engage an annular wall of a native heart valve when device 12 is positioned within an annulus of the native heart valve and device 12 is in the expanded configuration.

Device 12 includes the plurality of imaging markers 24, which includes two or more imaging markers including imaging marker 34. Imaging marker 34 is configured to aid in identifying device 12 within heart 14 via medical imaging. For example, imaging marker 34 may be configured to enhance an image of device 12 captured by an imaging system 60 (FIG. 3A), such that an approximate location of device 12 within heart 14 may be assessed by a clinician. In examples, the plurality of imaging markers 32 (e.g., imaging markers 34, 35, 36) may be located at one or more fixed locations on device 12, such that an approximate orientation of device 12 within heart 14 may be assessed using imaging system 60. In examples, as will be discussed, imaging marker 34 is configured to reflect and or otherwise interact with energy transmitted (e.g., the energy E (FIG. 3A)) by imaging system 60 to enhance a visual distinction from one or more structures of the heart 14 in a resulting image produced by imaging system 60. The visual distinction enabled by imaging marker 34 may assist a clinician in assessing a position and/or orientation of device 12 during and/or following a deployment of device 12 within heart 14.

In examples described herein, device 12 is configured to mechanically support imaging marker 34 such that imaging marker 34 is displaced from (e.g., substantially does not contact) tissue when device 12 is in the expanded configuration and positioned within an annulus of a native heart valve. Displacing imaging marker 34 may increase a visibility (e.g., an echogenic visibility) of imaging marker 34 in a resulting image of device 12 and portions of heart 14 produced by imaging system 60. For example, because an ultrasound image may display tissue in the same color (e.g., white) as imaging markers 34, 35, 36, imaging marker 34 configured to displace from tissue (e.g., not contact tissue) when device 12 is positioned or being positioned within heart 14 may help imaging marker 34 stand out from other structures of heart 14 (e.g., contrast with blood and/or tissues, which may be dark or black in color) on the resulting image (e.g., an ultrasound image).

In some examples, device 12 is be configured to mechanically support imaging marker 34 such that at least some portion of imaging marker 34 is displaced from other portions of device 12 (e.g., anchoring member 40, valve support 38, fixation elements 58, and others) when device 12 is in the expanded configuration. This may assist the visibility (e.g., echogenic visibility) of imaging marker 34 when the other parts (e.g., anchoring member 40, valve support 38, fixation elements 58) have limited visibility in a resulting image generally, or when the other parts have limited visibility when positioned near (e.g., adjacent to) a tissue wall.

In examples, device 12 is configured to limit a radial extension (e.g., relative to valve axis L) of imaging marker 34 at least when device 12 is in the expanded configuration For example, device 12 may be configured to mechanically support imaging marker 34 such that a radial extension of imaging marker 34 beyond anchoring member 40 is substantially limited or even prevented. In some examples, device 12 is configured to mechanically support imaging marker 34 such that imaging marker 34 do not extend radially beyond anchoring member 40 (e.g., beyond a perimeter defined by anchoring member 40) when device 12 is positioned within the annulus of a native heart valve. This may assist in displacing imaging marker 34 from tissue of heart 14 when device 12 is in the expanded configuration and positioned within an annulus of a native heart valve.

In some examples, imaging marker 34 extends from anchoring member 40 and/or valve support 38. In some examples, imaging marker 34 is configured to extend from device 12 (e.g., from anchoring member 40 and/or valve support 38) in the upstream direction of device 12 (e.g., a direction substantially opposite the arrows BF) at least when device 12 is in the expanded configuration. In addition to or instead of the upstream direction, in some examples, imaging marker 34 or another imaging marker 35, 36 is configured to extend from device 12 (e.g., from anchoring member 40 and/or valve support 38) in a downstream direction of device 12 (e.g., a direction substantially opposite the arrows BF) at least when device 12 is in the expanded configuration. Device 12 may mechanically support imaging marker 34 on any portion of device 12. For example, device 12 may mechanically support imaging marker 34 such that imaging marker 34 is laterally positioned (e.g., positioned relative to a point on axis L) substantially upstream (relative to the arrows BF) of inflow region 44, laterally positioned substantially downstream of outflow region 46, and/or laterally positioned substantially between inflow region 44 and outflow region 46.

In examples, device 12 mechanically supports the plurality of imaging markers 32 (e.g., imaging markers 34, 35, 36) such that imaging markers 34, 35, 36 are spatially positioned around a perimeter defined by device 12. For example, device 12 may mechanically support imaging markers 34, 35, 36 such that imaging markers 34, 35, 36 are spatially positioned around a perimeter P1 defined by anchoring member 40. Device 12 may mechanically support imaging markers 34, 35, 36 such that imaging markers 34, 35, 36 are spatially positioned around a perimeter P2 defined by valve support 38. In examples, device 12 is configured such that the defined perimeter (e.g., perimeter P1 and/or perimeter P2) is substantially surrounded by the annular wall of a native heart valve when fixation structure 56 grips the annular wall. In examples, device 12 defines the perimeter around the flow path provided by first sealing member 52, second sealing member 54, and valve assembly 42. In examples, device 12 defines the perimeter around valve axis L. The spatial positioning of imaging markers 34, 35, 36 around a defined perimeter (e.g., perimeter P1 and/or perimeter P2) may assist a clinician in assessing the orientation of device 12 relative to other structures of the heart when the clinician views an image of device 12 (e.g., an image generated by imaging system 60) within heart 14.

Device 12 may define the perimeter around any portion of device 12. In examples, the perimeter is a closed boundary extending around some portion of device 12 (e.g., anchoring member 40 and/or valve support 38). In some examples, the perimeter is an outer perimeter (along an outer surface of device 12). In some examples, the defined perimeter is a substantially planar perimeter, such that the perimeter substantially lies within a geometric plane (e.g., lies in the geometric plane or nearly fully lies in the geometric plane to the extent permitted by manufacturing tolerances). In some examples, the defined perimeter is a nonplanar perimeter, such that portions of the defined perimeter may lie in different geometric planes. In some examples, the defined perimeter is substantially perpendicular to valve axis L.

In some examples, device 12 is configured to mechanically support imaging markers 34, 35, 36 in a manner enabling a reduction in a length of device 12 in the delivery configuration and/or the expanded configuration. For example, device 12 may enable a reduction in length compared to a prosthetic device configured to extend a brim supported by and/or substantially encircling anchoring member 40, valve support 38, or another portion of device 12. In examples, device 12 mechanically supports one or more of imaging markers 34, 35, 36 such that imaging markers 34, 35, 36 extend from a defined perimeter (e.g., perimeter P1 and/or perimeter P2) in an upstream direction of device 12 (e.g., extend in a direction substantially opposite the arrows BF). In some examples, one or more of imaging markers 34, 35, 36 (e.g., imaging marker 36) includes a fixed end 62 mechanically coupled to a portion of device 12 (e.g., anchoring member 40, valve support 38, or another portion) and a free end 64 substantially opposite the fixed end. Device 12 may mechanically support the imaging marker such that free end 64 extends away from fixed end 62 in the upstream direction of device 12 to, for example, limit a radial extension of the imaging marker. In some examples, device 12 mechanically supports one or more of imaging markers 34, 35, 36 such that an imaging marker extends from a defined perimeter (e.g., perimeter P1 and/or perimeter P2) in a downstream direction of device 12 (e.g., extends in a direction substantially the same as the arrows BF). Device 12 may mechanically support the imaging marker such that the free end extends away from the fixed end in a downstream direction of device 12.

In examples, anchoring member 40 includes a plurality of anchoring member struts 66, such as anchoring member strut 68 and anchoring member strut 70. In examples, anchoring member struts 68, 70 are configured to urge device 12 (e.g., anchoring member 40) from the delivery configuration to the expanded configuration when an element constraining the radial expansion of device 12 (e.g., capsule 18 (FIGS. 1A-1B)) is at least partially displaced from device 12. Anchoring member struts 68, 70 may be resiliently biased to expand radially outward from valve axis L. In examples, anchoring member struts 68, 70 are configured to urge device 12 from the delivery configuration to the expanded configuration to cause device 12 to substantially fit into the annulus of a native heart valve. In examples, anchoring member struts 68, 70 are elongated members configured to substantially define a shape of anchoring member 40 in the expanded configuration and/or delivery configuration. In some examples, anchoring member struts 68, 70 may be joined to define one or more cells such as cell 72. For example, anchoring members 68, 70 may be joined to define one or more acute angles and/or obtuse angles defining cell 72. In examples, cell 72 is a substantially diamond-shaped cell. Anchoring member struts 68, 70 may be configured such that a size of cell 72 is variable, such that an expansion of cell 72 allows anchoring member struts 68, 70 to expand radially outward as device 12 transitions from the delivery configuration to the expanded configuration. In examples, anchoring member struts 68, 70 mechanically support other portions of device 12, such as fixation structure 56, second sealing member 54, and/or other portions of device 12. In examples, device 12 is configured such that the plurality of anchoring member struts 66 substantially surround valve axis L, valve support 38, valve assembly 42, and/or valve axis L.

Valve support 38 may include a plurality of valve support struts 74, such as valve support strut 76 and valve support strut 78. Valve support struts 76, 78 may be substantially elongated members configured to substantially define a shape of valve support 38 (e.g., in the expanded configuration and/or delivery configuration). In some examples, valve support struts 76, 78 may be joined to define one or more cells such as cell 80. For example, valve support struts 76, 78 may be joined to define one or more acute angles and/or obtuse angles defining cell 80. In examples, cell 80 is a substantially diamond-shaped cell. Valve support struts 76, 78 may be configured to mechanically support other portions of device 12, such as valve assembly 42, second sealing member 54, and/or other portions of device 12.

In examples, device 12 is configured such that valve support 38 and/or anchoring member 40 surround valve axis L at when device 12 is in the expanded configuration. In examples, valve support 38 includes a substantially tubular member (e.g., tubular or nearly tubular to the extent permitted by manufacturing tolerances). Valve support 38 (e.g., valve support struts 76, 78) may define one or more upstream crowns 82 at a first end 84 of valve support 38 (“first valve support end 84”). Valve support 38 (e.g., valve support struts 76, 78) may define one or more downstream crowns 86 defining a second end 88 of valve support 38 (“second valve support end 88”) opposite first valve support end 84. Anchoring member 40 (e.g., anchoring member struts 68, 70) may define one or more upper crowns 90 defining a first end 92 of anchoring member 40 (“first anchoring end 92”). Anchoring member 40 (e.g., anchoring member struts 68, 70) may include one or more lower crowns 94 defining a second end 96 of anchoring member 40 (“second anchoring end 96”) opposite first anchoring end 92. Anchoring member 40 may include and/or mechanically support fixation structure 56. Fixation structure 56 may include a first portion 102 (“first fixation portion 102”) and a second portion 104 (“second fixation portion 104’). Fixation structure 56 may be configured such that first fixation portion 102 is substantially between first anchoring end 92 and second fixation portion 104. Fixation structure 56 may be configured such that second fixation portion 104 is substantially between second anchoring end 96 and first fixation portion 102. In examples, fixation structure 56 (e.g., first fixation portion 102 and/or second fixation portion 104) defines a substantially annular shape surrounding valve axis L. In examples, fixation structure 56 defines a substantially annular shape surrounding valve support 38 and/or valve assembly 42.

In examples, the perimeter P1 is a perimeter defined by one or more portions of device 12. For example, perimeter P1 may be defined by one or more first anchoring ends such as first anchoring end 92, one or more upper crowns such as upper crown 194, and/or one or more anchor support struts such as anchoring member struts 68, 70. Device 12 may define perimeter P1 by mechanically supporting the one or more first anchoring ends, the one or more upper crowns, and/or the one or more anchor support struts such that the one or more first anchoring ends, the one or more upper crowns, and/or the one or more anchor support struts position on the perimeter P1. In some examples, the perimeter P1 may be a substantially contiguous component of device 12 (e.g., a component at least partially surrounding valve axis L). In some examples, perimeter P1 may be defined by one or more second anchoring ends such as second anchoring end 96 and/or one or more lower crowns such as lower crown 196.

In examples, the perimeter P2 is a perimeter defined by one or more portions of device 12. For example, perimeter P2 may be defined by one or more first valve support ends such as first valve support end 84, one or more upstream crowns such as upstream crown 190, and/or one or more valve support struts such as valve supports struts 76, 78. Device 12 may define perimeter P2 by mechanically supporting the one or more first valve support ends and/or the one or more upstream crowns such that the one or more first valve support ends and/or the one or more upstream crowns position on the perimeter P2. In some examples, the perimeter P2 may be a substantially contiguous component of device 12 (e.g., a component at least partially surrounding valve axis L). Device 12 may define the perimeter P1 by mechanically supporting one or more other portions of device 12 such that the other portions of device 12 position on the perimeter P1, such as other portions of valve support 38. Device 12 may define the perimeter P2 by mechanically supporting one or more other portions of device 12 such that the other portions of device 12 position on the perimeter P2, such as other portions of anchoring member 40 and/or fixation structure 56. In some examples, perimeter P1 may be defined by one or more second anchoring ends such as second anchoring end 96 and/or one or more lower crowns such as lower crown 196. In some examples, perimeter P2 may be defined by one or more second valve support ends such as second valve support end 88 and/or one or more downstream crowns such as downstream crown 192.

In examples, imaging marker 34 extends from upper crowns 90 of anchoring member 40. Additionally or alternatively, in some examples, imaging marker 34 may extend from upstream crown 190 of valve support 38. In some examples, imaging marker 34 may extend from other portions of device 12, such as from downstream crown 192, lower crown 196, a suitable location on valve support 38 between first valve support end 84 and second valve support end 88, a suitable location on anchoring member 40 between first anchoring end 92 and second anchoring end 96, or some other portion of device 12. As discussed previously, device 12 may be configured to expand radially from a delivery configuration to an expanded configuration when deployed at a target site within heart 14 (FIG. 1A-1B). Other states, representing stages of partial deployment, may exist between the delivery configuration and the expanded configuration, such as a partially expanded state wherein capsule 18 (FIG. 1A-1B) is positioned to allow an initial radial expansion of device 12 while continuing to constrain device 12 from further expansion.

In examples, anchoring member 40 includes a base 106 attached to outflow region 46 of valve support 38. In some examples, second anchoring end 96, second valve support end 88, downstream crowns 86, and/or lower crowns 94 are joined with and/or define base 106. In examples, the plurality of anchoring member struts (e.g., anchoring member struts 68, 70) define a plurality of arms 108 projecting radially outward (relative to valve axis L) from base 106. Fixation structure 56 may extend from arms 108. In examples, fixation structure 56 is configured such that, when device 12 is in the expanded configuration (as shown, for example, in FIGS. 2, 3A, 3B), fixation structure 56 and/or anchoring member 40 is spaced radially outward apart from valve support 38 by the gap G. When prosthetic heart valve is in a delivery configuration (FIG. 1A), gap G may be reduced or substantially eliminated. In examples, fixation structure 56 includes a ring (e.g., a cylindrical or conical ring). Fixation structure 56 may define an engagement surface 110 configured to press outwardly against the native annulus. In examples, engagement surface 110 mechanically supports a plurality of fixation elements 58 projecting radially outward from engagement surface 110. In examples, one or more of fixation elements 58 may be inclined toward an upstream direction (e.g., inclined in a direction from outflow region 46 to inflow region 44). The fixation elements 58, for example, can be barbs, hooks, cleats, tines, or other elements configured to engage tissue when device 12 is in the expanded configuration within heart 14.

In examples, device 12 is configured such that first valve support end 84 is displaced by a displacement C from first anchoring end 92 when device 12 is in the expanded configuration. The displacement C may be substantially parallel to the valve axis L. In examples, first valve support end 84 is displaced from first anchoring end 92 in a direction from outflow region 46 toward inflow region 44. In some examples, first valve support end 84 is displaced from first anchoring end 92 in a direction from inflow region 44 toward outflow region 46. The displacement C may be any displacement. In some examples, as indicated in FIG. 3A, the displacement C may be substantially zero (subject to manufacturing and/or other tolerances), such that first valve support end 84 is substantially even with first anchoring end 92 when device 12 is in the expanded configuration.

In examples, anchoring member 40 includes a smooth bend 112 (FIG. 3A) defining a transition between arms 108 and fixation structure 56. In examples, second fixation portion 104 extends from arms 108 substantially at smooth bend 112. Arms 108 and fixation structure 56 can be formed integrally from a continuous strut or support element such that smooth bend 112 is a bent portion of the continuous strut. In other embodiments, smooth bend 112 may be a separate component with respect to either the arms 108 or the fixation structure 56. For example, smooth bend 112 may be attached to arms 108 and/or fixation structure 56 using a weld, adhesive or other technique. In examples, smooth bend 112 is configured to ease a recapture of device 12 by capsule 18 (FIG. 2A-2B) or other container after the device 12 has been at least partially deployed.

First sealing member 52 is configured to limit (e.g., substantially reduce) blood flow in a radial direction (e.g., substantially perpendicular to valve axis L) through valve support 38. Second sealing member 54 is configured to limit blood flow in a radial direction (e.g., substantially perpendicular to valve axis L) through anchoring member 40. In examples, first sealing member 52 and/or second sealing member 54 may be made from a flexible material, such as Dacron® or another type of polymeric material. First sealing member 52 may substantially cover (e.g., be in contact with) an interior surface of valve support 38 facing valve axis L and/or an exterior surface of valve support 38 facing away from valve axis L. Second sealing member 54 may substantially cover (e.g., be in contact with) an interior surface of anchoring member 40 facing valve axis L and/or an exterior surface of anchoring member 40 facing away from valve axis L. In some examples, first sealing member 52 is attached to valve assembly 42. In examples, second sealing member 54 is attached to anchoring member 40 such that fixation elements 58 are uncovered by second sealing member 54, such that, for example, fixation elements 58 may engage tissue when device 12 is in the expanded configuration.

Device 12 may be configured to replace any native heart valve. In examples, device 12 may be configured to replace a previously implanted prosthetic heart valve. In examples, valve assembly 42 includes one or more leaflets configured to control a flow of blood through device 12, such as leaflet 43. Valve assembly 42 may comprise any suitable number of leaflets (e.g., three leaflets for a prosthetic tricuspid valve).

As discussed, imaging marker 34 may function to provide imaging feedback (e.g., echogenic feedback) to imaging system 60 (e.g., an ultrasound probe). In examples, imaging marker 34 is configured (e.g., sized) to enhance the imaging feedback. For example, when imaging system 60 is an ultrasound probe, imaging marker 34 may be configured to define one or more surfaces defining a dimension extending over at least one wavelength of an acoustic wave generated by the ultrasound probe. In some cases, imaging marker 34 is configured to define one or more surfaces defining a dimension extending at least 10 wavelengths of the acoustic probe. Defining one or more such surfaces may improve echogenic visibility without substantial length (e.g., length substantially parallel to valve axis L) to device 12. In some examples, imaging marker 34 may be configured to define a dimension extending over at least one wavelength of a wave (e.g., an acoustic wave) having a frequency between about 4 and 5 megahertz (MHz). In examples, where a frequency of an acoustic wave is between 4 and 5 MHz, the wavelength of the acoustic wave may be in the range of about 0.31 to 0.39 millimeters (mm), with blood as the medium through which the acoustic waves are traveling. Accordingly, in some examples, imaging marker 34 may define a length D, where D is between about 2.0 and 5.0 mm. In some examples, length D may be between 3.0 and 4.0 mm, inclusive of 3.0 mm and 4.0 mm. In some examples, length D may be between 3.1 and 3.9 mm, inclusive of 3.1 and 3.9 mm.

In examples, imaging marker 34 is configured to present angles to imaging system 60 to enhance a resulting image. The displayed angles may enhance an image generated by an ultrasound probe as sound waves leave the ultrasound probe, reflect from imaging marker 34, and return to the ultrasound probe to generate an image. For example, imaging system 60 may be a transesophageal probe (e.g., positioned in the esophagus of a patient) such as an ultrasound probe. In examples, imaging marker 34 may be shaped to increase the imaging feedback (e.g., increase an echogenic signal returned by imaging marker 34) by including surfaces 114, 116, 118, and/or 120. In examples, surface 114 and surface 116 are portions of a first contiguous surface defining an angle (e.g., an acute angle). In examples, surface 118 and surface 120 are portions of a second contiguous surface defining an angle (e.g., an obtuse angle).

In some examples, surfaces 114 and 116 may be disposed at an angle to each other (e.g., a 90 degree angle), giving an angular shape to imaging marker 34. The angularly-shaped imaging marker 34 may enhance echogenic visibility when struck at a suitable angle by acoustic waves from imaging system 60 (e.g., an ultrasound probe). In some examples, imaging marker 34 is configured to such that one or more surfaces of surfaces 114, 116, 118, 120 is substantially perpendicular (e.g., perpendicular or nearly perpendicular) to sound waves generated by an ultrasound probe (e.g., a transesophageal probe) when device 12 is located within heart 14 (FIG. 1A-1B).

In examples in which imaging system 60 is a transesophageal probe and device 12 is positioned within or in the vicinity of a tricuspid valve of heart 14, device 12 may be configured to mechanically support imaging marker 34 such that sound waves travel from imaging system 60 to imaging marker 34 at about a 30 to 60 degree angle (e.g., a 45 to 50 degree angle) to the sagittal plane of the patient. Device 12 may be configured such that one or more flat surfaces of surfaces 114, 116, 118, 120 provide a flat surface substantially perpendicular to the energy E transmitted by imaging system 60. This may enhance the echogenic visibility of imaging marker 34 as one or more of surfaces 114, 116, 118, 120 reflect energy (e.g., acoustic energy) back to imaging system 60

In some examples, as illustrated in FIG. 4, in some examples, imaging marker 34 define a curved shape, such as a c-shape. Curved imaging markers may enhance visibility (e.g., echogenic visibility) when struck by energy (e.g., acoustic waves) generated by imaging system 60 (e.g., an ultrasound probe) over a wider variety of angles, because. For example, some portion of a surface of the curved imaging marker may be more likely to be at a perpendicular angle to energy transmitted by imaging system 60. Hence, the curved imaging marker may be more likely to interact with sound waves generated by an ultrasound probe and reflect the sound waves back to the ultrasound probe. In some examples, the curved imaging markers may include one or more surfaces (e.g., one or more of surface 114, surface 116, surface 118, and/or surface 120) defining a concavity. In some examples, the curved imaging marker may include one or more surfaces (e.g., one or more of surface 114, surface 116, surface 118, and/or surface 120) defining a convexity. The curved imaging marker may have a generally curved and/or curvilinear shape in addition to or instead of one or more sharp corners, so that many angles can be presented to imaging system 60.

Imaging marker 34 may be configured such that at least some portion of imaging marker 34 extends inward toward valve axis L when device 12 mechanically supports imaging marker 34. In examples, the portion of imaging marker 34 extends inward toward valve axis L relative to a fixed end of imaging marker 34 when device 12 mechanically supports imaging marker 34. In examples, one or more of one or more of surfaces 114-120 extends toward valve axis L relative to the fixed end when device 12 mechanically supports imaging marker 34. The portion of imaging marker 34 extending inward toward valve axis L may limit a radial extension (e.g., relative to valve axis L) of imaging marker 34 at least when device 12 is in the expanded configuration. In some examples, the portion of imaging marker 34 is configured such that a radial extension of imaging marker 34 beyond anchoring member 40 is substantially limited at least when device 12 is in the expanded configuration. In some examples, the portion of imaging marker 34 is configured such that imaging marker 34 does not extend radially beyond anchoring member 40 at least when device 12 is in the expanded configuration. This may assist in displacing imaging marker 34 from tissue of heart 14 when device 12 is in the expanded configuration and positioned within an annulus of a native heart valve.

FIG. 4 is a partial side view of device 12 example device 12 mechanically supporting a plurality of imaging markers 32, including imaging marker 34 and imaging marker 35. Imaging marker 34 and imaging marker 35 are arranged around perimeter P1 defined by anchoring member 40. A location of the perimeter P1 defined by device 12 is illustrated with a dashed line. Imaging marker 34 and imaging marker 35 are each discrete image markers configured to interact with the transmitted energy of imaging system 60 (FIG. 3A). Device 12 mechanically supports imaging marker 34 and imaging marker 35 such that imaging marker 34 and imaging marker 35 are separated by an arc-length S1 on perimeter P1. The arc-length S1 may be defined by any subtending angle (e.g., a subtending angle with a vertex substantially on valve axis L). For example, the subtending angle may be about 30 degrees, about 45 degrees, about 90 degrees, or be some other subtending angle.

In examples, each individual imaging marker in the plurality of image markers 24 (regardless of a location of the individual imaging marker on device 12) may define a closed boundary defining one or more spatial parameters of the individual image marker. The spatial parameter may be, for example, a length, a surface area, a volume, and/or another spatial parameter. For example, imaging marker 34 may define a closed boundary B1 defining one or more the spatial parameters of imaging marker 34, Imaging marker 35 may define a closed boundary B2 defining one or more spatial parameters of imaging marker 35. Individual imaging markers (e.g., imaging markers 34, 35, 36) may be mechanically supported by device 12 such that each closed boundary of an image marker is displaced from every other closed boundary of an image marker. The plurality of imaging markers 32 spatially arranged on device 12 (e.g., on perimeter P1 and/or perimeter P2) may enhance the ability of a clinician to assess the position and/or orientation of device 12 within heart 14 using an image generated by imaging system 60.

Device 12 may be configured to mechanically support imaging markers 34, 35, 36 on a defined perimeter (e.g., perimeter P1 and/or perimeter P2) such that imaging markers 34, 35, 36 are separated from an adjacent imaging marker positioned on the perimeter by any suitable subtending angle. In examples, one of the plurality of imaging markers 32 may extend from each upper crown, lower crown, upstream crown, and/or downstream crown of device 12. In some examples, a first imaging marker may extend from a first crown on a perimeter and a second imaging marker may extend from a second crown on the perimeter, and one or more additional crowns on the perimeter which do not support an imaging marker may be between the first crown and the second crown. In other words, any two adjacent markers (e.g., imaging marker 34 and imaging marker 35) may be spaced apart from each other by one or more crowns that do not mechanically support an imaging marker.

In some examples, device 12 is configured to mechanically support imaging markers 34, 35, 36 such that imaging markers 34, 35, 36 are spaced apart at substantially equal intervals about a defined perimeter of device 12 (e.g., symmetrically, such that each imaging member on a perimeter defines a substantially equal subtending angle with an adjacent imaging member on the perimeter). In some examples, device 12 is configured to mechanically support imaging markers 34, 35, 36 such that a first spacing between an imaging marker on the perimeter and a first adjacent imaging marker on the perimeter is different from a second spacing between the imaging marker and a second adjacent imaging marker on the perimeter (e.g., when the imaging marker is between the first adjacent imaging marker and the second adjacent imaging marker). Hence, imaging markers 34, 35, 36 may be spaced apart at any suitable interval.

In some examples, reducing the interval by which adjacent imaging markers are spaced apart, and/or including more imaging markers about a defined perimeter of device 12, may reduce a need to rotate device 12 about valve axis L in order to achieve sufficient visibility (e.g., echogenic visibility) of device 12. In examples, imaging markers 34, 35, 36 may be spaced apart from an adjacent imaging marker to define a subtending angle between approximately 10 and 90 radial degrees. In some examples, imaging markers 34, 35, 36 are spaced apart from an adjacent imaging marker to define a subtending angle of about 15 degrees, about 30 degrees, about 45 degrees, or some other subtending angle.

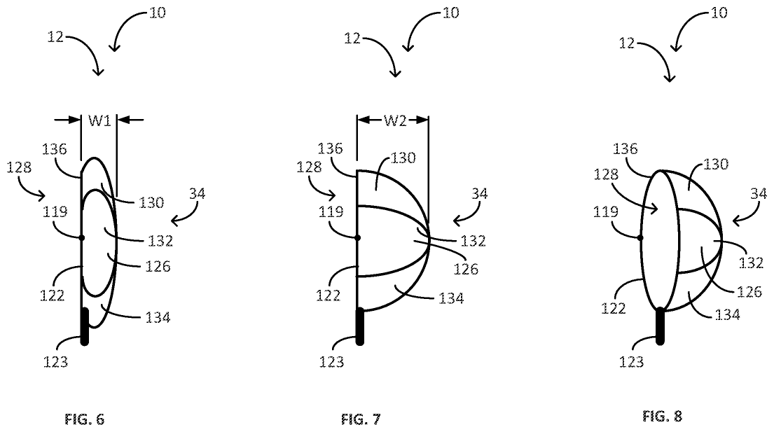

In examples, imaging marker 34 is configured to transition from a compressed state to an extended state when device 12 transitions from the delivery configuration to the expanded configuration. Imaging marker 34 may be configured to increase a displacement in a particular direction (e.g., a direction substantially perpendicular to valve axis L) when imaging marker 34 transitions from the compressed state to the extended state. For example, FIG. 6 illustrates a plan view of imaging marker 34 in a compressed state, with imaging marker 34 defining a displacement W1 from a fixed point 119 on a base section 122 of imaging marker 34. FIG. 7 illustrates a plan view of imaging marker 34 in an extended state, with imaging marker 34 defining a displacement W2 from fixed point 119 on base section 122 of imaging marker 34 The displacement W2 is greater than the displacement W1. Imaging marker 34 is supported by a portion 123 of device 12, such as a portion of anchoring member 40, a portion of valve support 38, or some other portion of device 12. FIG. 8 illustrates a perspective view of imaging marker 34 in the extended state.

In examples, device 12 is configured to substantially establish the compressed state when imaging marker 34 is constrained from expansion (e.g., by capsule 18 and/or another portion of device 12 when device 12 is in the delivery configuration (FIG. 1A)). In examples, imaging marker 34 is configured to establish the extended state when imaging marker 34 is unconstrained from expansion (e.g., when capsule 18 and/or another portion of device 12 is displaced from imaging marker 34 (FIG. 1B)). In examples, device 12 is configured to mechanically support imaging marker 34 such that, when device 12 is in an expanded configuration, imaging marker 34 extends in a direction toward valve axis L. In examples, imaging marker 34 may be configured to establish the compressed state when device 12 is in the delivery configuration. In examples, imaging marker 34 may be configured to establish the extended state when device 12 is in the expansion configuration (e.g., when capsule 18 is at least partially displaced from device 12 (FIG. 1B)).

Imaging marker 34 may be resiliently biased into the expanded state, such that imaging marker 34 transitions from the compressed state when imaging marker is constrained to the expanded state when imaging marker 34 is unconstrained. Hence, imaging marker 34 may be configured to establish the compressed state when capsule 18 constrains device 12 allowing, for example, a decrease in a cross-sectional dimension of capsule 18. Imaging marker 34 may be configured to extend when capsule 18 displaces at least partially from device 12 allowing, for example, imaging marker 34 to present surfaces to imaging system 60 in an orientation enhancing a visibility (e.g., echogenic visibility) to imaging system 60.

Imaging marker 34 may include a body section 124 defining a surface 126. Body section 124 may be coupled to base section 122. In examples, base section 122 defines a continuous surface extending across a perimeter 136 defined by body section 124. In examples, body section 124 is configured such that surface 126 defines a substantially hemispherical surface at least when imaging marker 34 is in the extended state. Body 213 may define surface 126. In some examples, at least body 214 defines a boundary of a cell 128 configured to allow imaging marker 34 to transition between the compressed state and the extended state.

In examples, body section 124 may include one or more panels, such as panel 130, panel 132, and/or panel 134. Panels 130, 132, 134 may be coupled to base section 122 (e.g., coupled to perimeter 136). In examples, panels 130, 132, 134 are configured to define displacement W1 when imaging marker 34 is in the compressed state. Panels 130, 132, 134 may be configured to define displacement W2 when imaging marker 34 is in the extended state. In examples, panels 130, 132, 134 are configured to flex inward (e.g., toward point 119) when imaging marker 34 is in the compressed state. Panels 130, 132, 134 may be configured to flex outward (e.g., away from point 119) when imaging marker 34 transitions from the compressed state to the expanded state. In examples, panels 130, 132, 134 are resiliently biased to flex outward to define the dimension W2 when imaging marker 34 is in the extended state. Panels 130, 132, 134 may be formed by cutting (e.g., laser cutting) body section 124, coupling panels 130, 132, 134 to base section 122 by welding, soldering, or some other joining method, or coupled to base section 122 using another suitable technique.

In some examples, imaging marker 34 defines a substantially smooth surface finish configured to provide a substantially specular reflection when imaged by imaging system 60 (FIG. 3A). The smooth surface finish may be a surface finish configured to reflect incident energy (e.g., an echogenic sound wave) in substantially a single direction from the surface. The smooth surface finish may enhance the return of transmitted energy (e.g., acoustic energy) to imaging system 60. The surface may smoothed by polishing, grinding, other abrasive methods, and/or other surface smoothing techniques.

In other examples, in addition to or instead of a smooth surface finish, all or part of imaging marker 34 defines a substantially rough surface finish configured to provide a substantially diffuse reflection when imaged by imaging system 60. The rough surface finish may be a surface finish configured to reflect incident energy (e.g., an echogenic sound wave) at many angles from the surface. The rough surface may enhance the return of transmitted energy (e.g., acoustic energy) to imaging system 60 over a variety of imaging angles between the surface and imaging system 60. The surface may be roughened by bead blasting, sandblasting, media blasting, and/or other surface roughening techniques. In some examples, the plurality of imaging markers 32 may include a combination of both smooth surface finishes and rough surface finishes.

In examples, anchoring member 40 (e.g., anchoring member struts 68, 70) and/or valve support 38 (e.g., valve support struts 76, 78) comprises a metal and/or other material defining a first cross-sectional dimension (e.g., a first cross-sectional dimension substantially perpendicular to valve axis L). Imaging marker 34 may comprise a metal and/or other material defining a second cross-sectional dimension (e.g., a second cross-sectional dimension substantially perpendicular to valve axis L). The second cross-sectional dimension may be less than, equal to, or higher than the first cross-sectional dimension. In some examples, imaging marker 34 is substantially thinner than at least some portion of anchoring member 40 and/or at least some portion of valve support 38. Imaging marker 34 may be made thinner than the portions of anchoring member 40 and/or valve support 38 by grinding, swaging, other thinning or stretching processes, or attaching imaging marker 34 to anchoring member 40 or valve support 38 as a substantially separate component. In some examples, imaging marker 34 may be sewn to anchoring member 40 or valve support 38 (e.g., using a supporting fabric). In some examples, imaging marker 34 may be formed separately and riveted to anchoring member 40 or valve support.

In some examples, imaging marker 34 is substantially integral with anchoring member 40 or valve support 38. Imaging marker 34 may be made out of the same material or materials as anchoring member 40 and/or valve support 38. In some examples, imaging marker 34 comprises nitinol. In some examples, imaging marker 34 is a shaped nitinol extension. Imaging marker 34 may comprise other biocompatible materials configured to provide imaging feedback (e.g., echogenic feedback) to imaging system 60 (e.g., metals, alloys, synthetic materials, or the like).

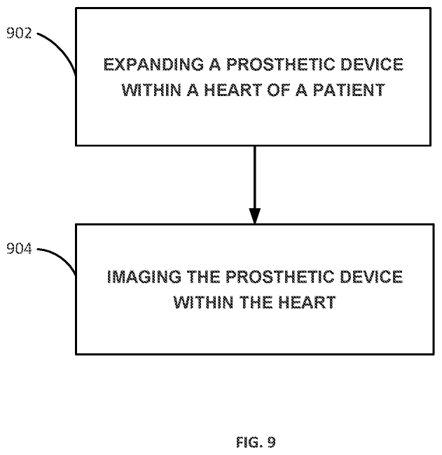

An example technique for positioning a prosthetic device within a heart of a patient is illustrated in FIG. 9. Although the technique is described mainly with reference to device 12 of FIGS. 1-8, the technique may be applied to other prosthetic devices in other examples.

The technique includes expanding a prosthetic device 12 within a heart of a patient (902). Prosthetic device 12 may be in proximity to an annulus of a heart valve of heart 14. In examples, prosthetic device 12 expands from a delivery configuration defining a first displacement from a valve axis L defined by prosthetic device 12 to an expanded configuration defining a second displacement from the valve axis L, wherein the second displacement is greater than the first displacement. In examples, prosthetic device 12 is positioned with a capsule 18 in the delivery configuration, and capsule 18 is displaced from prosthetic device 12 to expand to the expanded configuration. In examples, capsule 18 is displaced by a delivery catheter 16 coupled to capsule 18. In some examples, capsule 18 and prosthetic device 12 are delivered to the heart using a guide catheter 20 defining a lumen configured to allow capsule 18 and prosthetic device 12 to pass therethrough.

Prosthetic device 12 may include an anchoring member 40 configured to engage the annulus of the heart valve when prosthetic device 12 expands from the delivery configuration to the expanded configuration. Anchoring member 40 may expand when prosthetic device 12 expands from the delivery configuration to the expanded configuration. In examples, anchoring member 40 causes the valve axis L to pass through the annulus when anchoring member 40 engages the annulus. In examples, anchoring member 40 includes a fixation structure 56 including one or more fixation elements 58. Anchoring member 40 may engage the annulus by causing fixation structure 56 to engage the annulus.

Anchoring member 40 may position a valve support 38 substantially within the annulus when anchoring member 40 engages the annulus. Valve support 38 may define a flow path for a blood flow through prosthetic device 12 from an inflow region 44 of valve support 38 to an outflow region 46 of valve support 38. Valve support 38 may mechanically support a valve assembly 42 configured to allow the blood flow through the flow path. In examples, prosthetic device 12 defines a downstream direction from inflow region 44 to outflow region 46. Prosthetic device 12 may define an upstream direction from outflow region 46 to inflow region 44. Valve assembly 42 may allow blood to flow through device 12 in at least the downstream direction. In examples, valve assembly 42 may substantially prevent blood from flowing through device 12 in the upstream direction.

Prosthetic device 12 may support a plurality of imaging markers 32 (e.g., imaging markers 34, 35, 36) around a perimeter defined by prosthetic device 12 (e.g., when prosthetic device 12 is in the expanded configuration). The defined perimeter may surround valve axis L. In examples, the defined perimeter surrounds the flow path defined by valve support 38. The perimeter may be defined by any portion of prosthetic device 12. In examples, anchoring member 40 defines at least some portion of the perimeter (e.g., perimeter P1). In examples, valve support 38 defines at least some portion of the perimeter (e.g., perimeter P2).

The technique includes imaging prosthetic device 12 within heart 14 using an imaging system 60 (904). Prosthetic device 12 may support imaging marker 34 such that imaging marker 34 presents one or more angles and/or curved surfaces to imaging system 60 when imaging system 60 transmits energy to capture an image of prosthetic device 12 within heart 14. Prosthetic device 12 may support imaging marker 34 such that imaging marker 34 presents one or more angles and/or curved surfaces to imaging system 60 when imaging system 60 is positioned in the esophagus of a patient. In examples, imaging system 60 is an ultrasound probe which transmits acoustic energy to capture the image. In examples, imaging marker 34 include surfaces 114, 116, 118, and/or 120 configured to defines an acute angle and/or obtuse angle. In some examples, imaging marker 34 may define a curved shape, such as a c-shape.

Prosthetic device 12 may support at least one imaging marker (e.g., imaging marker 34) such that at least some portion of imaging marker 34 is displaced from tissue of the heart when anchoring member 40 engages the annulus of the heart valve. In examples, imaging marker extends in a direction away from the defined perimeter to, for example, assist in displacing imaging marker 34 from the tissue. In examples, imaging marker 34 extends away from the defined perimeter in an upstream direction of device 12. In examples, imaging marker 34 extends away from the defined perimeter in a downstream direction of device 12. In examples, imaging marker 34 extends from the defined perimeter in a direction toward valve axis L. In examples, an angled, curved, or curvilinear portion of imaging marker 34 extends from the defined perimeter in a direction toward valve axis L.

In examples, prosthetic device 12 supports imaging markers 34, 35, 36 such that each of imaging markers 34, 35, 36 is separated from every other of imaging markers 34, 35, 36 by an arc-length S1 on the defined perimeter. The arc-length S1 may be defined by any subtending angle (e.g., a subtending angle with a vertex substantially on valve axis L). For example, the subtending angle may be about 30 degrees, about 45 degrees, about 90 degrees, or be some other subtending angle. In examples, each individual imaging marker in the plurality of image markers 24 defines a closed boundary defining one or more spatial parameters of the individual image marker. For example, imaging marker 34 may define a closed boundary B1. Imaging marker 35 may define a closed boundary B2. Device 12 may support the individual imaging markers such that each closed boundary of an image marker is displaced from every other closed boundary of an image marker.

Prosthetic device 12 may support imaging marker 34 such that imaging marker 34 presents one or more angles and/or curved surfaces to an imaging system 60 when imaging system 60 transmits energy to capture an image of prosthetic device 12 within heart 14. To enhance a resulting image. Prosthetic device 12 supports imaging marker 34 such that imaging marker 34 presents one or more angles and/or curved surfaces to imaging system 60 when imaging system 60 is positioned in the esophagus of a patient. In examples, imaging system 60 is an ultrasound probe which transmits acoustic energy to capture the image. In examples, imaging marker 34 include surfaces 114, 116, 118, and/or 120 configured to defines an acute angle and/or obtuse angle. In some examples, imaging marker 34 may define a curved shape, such as a c-shape.

Imaging marker 34 may transition from a compressed state to an extended state when device 12 transitions from the delivery configuration to the expanded configuration. Imaging marker 34 may increase a displacement in a particular direction (e.g., a direction substantially perpendicular to valve axis L) when imaging marker 34 transitions from the compressed state to the extended state. In examples, imaging marker 34 substantially establishes the compressed state when imaging marker 34 is substantially constrained from expansion (e.g., by capsule 18 and/or another portion of device 12). In examples, imaging marker 34 substantially establishes the extended state when imaging marker 34 is substantially unconstrained from expansion. In examples, device 12 supports imaging marker 34 such that, when device 12 is in an expanded configuration, imaging marker 34 extends in a direction toward valve axis L. Imaging marker 34 may include panels 130, 132, 134 coupled to a base section 122 which flex inward (e.g., toward fixed point 119) when imaging marker 34 is in the compressed state. Panels 130, 132, 134 may flex outward (e.g., away from fixed point 119) when imaging marker 34 transitions from the compressed state to the expanded state.

Various examples of the disclosure have been described. Any combination of the described systems, operations, or functions is contemplated. These and other examples are within the scope of the following claims.

1. A prosthetic device, comprising:
an anchoring member configured to engage an annulus of a heart valve of a heart, wherein the anchoring member is configured to cause a valve axis defined by the prosthetic device to pass through the annulus when the anchoring member engages the annulus;
a valve support mechanically supported by the anchoring member and surrounding the valve axis, wherein the valve support is configured to define a flow path from an inflow region of the valve support to an outflow region of the valve support;
a valve assembly mechanically supported by the valve support within the flow path, wherein the valve assembly is configured to allow a blood flow through the flow path; and
a plurality of imaging markers mechanically supported by the prosthetic device around a perimeter defined by the prosthetic device, wherein the perimeter surrounds the valve axis.

2. (canceled)

3. (canceled)

4. The prosthetic device of claim 1, wherein at least one imaging marker of the plurality of imaging markers extends from the defined perimeter in an upstream direction of the prosthetic device, wherein the upstream direction is a direction from the outflow region toward the inflow region.

5. The prosthetic device of claim 1, wherein at least one imaging marker of the plurality of imaging markers extends from the defined perimeter in a downstream direction of the prosthetic device, wherein the downstream direction is a direction from the inflow region toward the outflow region.

6. The prosthetic device of claim 1, wherein at least a portion of at least one imaging marker of the plurality of imaging markers extends from the defined perimeter in a direction toward the valve axis.

7.-16. (canceled)

17. The prosthetic device of claim 1, wherein the perimeter is a planar perimeter, and wherein the planar perimeter is substantially perpendicular to the valve axis.

18.-22. (canceled)

23. The prosthetic device of claim 1, wherein the anchoring member comprises a plurality of anchoring struts defining a plurality of upper crowns, wherein the plurality of upper crowns define at least a portion of the perimeter.

24. The prosthetic device of claim 1, wherein the valve support comprises a plurality of valve support struts defining a plurality of upstream crowns, wherein the plurality of upstream crowns define at least a portion of the perimeter.

25. The prosthetic device of claim 1, wherein at least one imaging marker of the plurality of imaging markers includes a first portion and a second portion and defines an angle between the first portion and the second portion.

26. The prosthetic device of claim 1, wherein at least one imaging marker of the plurality of imaging markers defines a curved surface.

27. The prosthetic device of claim 1, wherein at least one imaging marker of the plurality of imaging markers is substantially hemispherically shaped.

28. The prosthetic device of any of claims 1-27, wherein at least one imaging marker of the plurality of imaging markers is configured to define a compressed state when constrained and an extended state when unconstrained, wherein the at least one imaging marker is configured to increase a displacement in a particular direction when the at least one imaging marker transitions from the compressed state to the extended state.

29. The prosthetic device of claim 1, wherein at least one imaging marker of the plurality of imaging markers defines a surface defining a dimension of from about 2 millimeters to about 5 millimeters.

30.-33. (canceled)

34. A method, comprising:
delivering a prosthetic device to a heart of a patient in a radially compressed delivery configuration;
expanding the prosthetic device within the heart of the patient, the prosthetic device comprising:
an anchoring member configured to engage an annulus of a heart valve of the heart, wherein the anchoring member is configured to cause a valve axis defined by the prosthetic device to pass through the annulus when the anchoring member engages the annulus,
a valve support mechanically supported by the anchoring member and surrounding the valve axis, wherein the valve support is configured to define a flow path from an inflow region of the valve support to an outflow region of the valve support,
a valve assembly mechanically supported by the valve support within the flow path, wherein the valve assembly is configured to allow a blood flow through the flow path, and
a plurality of imaging markers mechanically supported by the prosthetic device around a perimeter defined by the prosthetic device, wherein the perimeter surrounds the valve axis; and
imaging the prosthetic device within the heart using an imaging system.

35. (canceled)

36. (canceled)

37. The method of claim 34, further comprising extending, by the prosthetic device, at least one imaging marker from the defined perimeter in an upstream direction of the prosthetic device, wherein the upstream direction is a direction from the outflow region toward the inflow region.

38. The method of claim 34, further comprising extending, by the prosthetic device, at least one imaging marker from the defined perimeter in a downstream direction of the prosthetic device, wherein the downstream direction is a direction from the inflow region toward the outflow region.

39. The method of claim 34, further comprising extending, by the prosthetic device, a portion of at least one imaging marker from the defined perimeter in a direction toward the valve axis.

40.-44. (canceled)

45. The method of claim 34, further comprising defining, by at least one of the imaging markers, angle between a first portion of the at least one imaging marker and a second portion of the at least one imaging marker.

46. The method of claim 34, further comprising defining, by at least one of the imaging markers, a curved surface.

47. The method of claim 34, wherein at least one imaging marker is configured to define a compressed state when constrained and an extended state when unconstrained, and further comprising increasing, by the at least one imaging marker, a displacement of the at least one imaging marker in a particular direction when the at least one imaging marker transitions from the compressed state to the extended state.

48. The method of claim 34, further comprising:
transmitting, using an ultrasound probe, acoustic wave energy to a heart when the prosthetic device is positioned within the heart; and
defining, by at least one of the imaging markers, a surface defining a spatial dimension greater than a wavelength of the acoustic wave energy transmitted to the heart.

49.-51. (canceled)
